(19) United States
(12) Patent Application Publication
(10) Pub. No.: US 2005/0081311 A1
(43) Pub. Date: Apr. 21, 2005

(54) Pearly-lustre coloring agents for keratin fibers

(76) Inventors: Juergen Schmenger, Weiterstadt (DE); Dirk Lauscher, Seeheim-Jugenheim (DE); Annelie Doehling, Muenster (DE); Helga Kreher, Muenster (DE)

Correspondence Address: Striker Striker & Stenby, 103 East Neck Road, Huntington, NY, 11743, US

(21) Appl. No.: 10/494,398

(22) PCT Filed: May 13, 2003
(86) PCT No.: PCT/EP03/04961

(30) Foreign Application Priority Data
Aug. 31, 2002 (DE) 102 40 276.0

(51) Int. Cl. A61K 7/13

(52) U.S. Cl. 8/405

(57) ABSTRACT

The colorant for dyeing keratin fibers contains oxidation dye precursors and/or direct dyes and a combination of from 2 to 12 percent by weight of at least one long-chain fatty alcohol, from 2 to 12 percent by weight of at least one alkanolamide, from 0.5 to 10 percent by weight of at least one fatty alcohol alkoxylate and from 0.5 to 10 percent by weight of at least one quatemary ammonium compound. The weight ratio of fatty alcohol to alkanolamide is from 0.5:1.5 to 1.5:0.5. A method of making the colorant is described.

The object of the invention are nacreous colorants for dyeing keratin fibers, particularly human hair, and containing direct dyes and/or oxidation dyes and a special raw material combination of long-chain fatty alcohols, alkanolamides, fatty alcohol alkoxylates and quaternary ammonium compounds, furthermore a method for dyeing keratin fibers by use of said colorants and the use of the aforesaid special raw material combination to impart a lasting nacreous luster to colorants.

Coloring preparations are usually in the form of aqueous—preferably thickened—solutions or emulsions and besides dyes contain, for example, fatty alcohols and/or other oil components, emulsifiers and surfactants and optionally alcohols. Oxidative colorants as a rule consist of two components: (i) the dye carrier composition containing the dyes and (ii) the oxidant preparation, said components being mixed with one another just before use and then applied to the fibers to be dyed. Such dye carrier compositions have no nacreous luster and, hence, nacreous luster-imparting additives are added to them. Moreover, such a nacreous luster effect disappears when the dye carrier composition and the oxidant are mixed.

DE 38 34 142 A1 discloses creamy hair colorants based on a complicated mixture of numerous raw materials including, among others, C₁₄-C₂₀ fatty alcohols, alkanolamides and fatty alcohol alkoxylates. These colorants, however, contain no quaternary ammonium salts and they do not exhibit a nacreous luster.

The goal was therefore to develop a dye carrier composition which by the interaction of just a few raw materials and without the addition of a nacreous luster-imparting additive would exhibit a lasting mother-of-pearl-like character (“nacreous effect”) which would remain unchanged even after the oxidant preparation and the dye carrier composition have been mixed. Another goal was to improve appreciably the care effect compared to that of the common, prior-art formulations after the dye composition is rinsed out.

Surprisingly, we have now found that this goal can be reached quite readily by use of a combination of at least one long-chain fatty alcohol, an alkanolamide, a fatty alcohol alkoxylate and a quaternary ammonium salt.

The object of the present invention is therefore a colorant for keratin fibers—particularly hair—which is based on oxidation dye precursors and/or direct dyes, said colorant containing a combination of
a) at least one long-chain fatty alcohol, preferably in an amount from 1 to 20 weight percent and particularly from 2 to 12 weight percent,
b) at least one alkanolamide, preferably in an amount from 1 to 20 weight percent and particularly from 2 to 12 weight percent,
c) at least one fatty alcohol alkoxylate, preferably in an amount from 0.1 to 15 weight percent and particularly from 0.5 to 10 weight percent, and
d) at least one quaternary ammonium compound (in the following referred to as “ammonium quat”), preferably in an amount from 0.1 to 15 weight percent and particularly from 0.5 to 10 weight percent,
   the weight ratio of fatty alcohol (a) to alkanolamide (b) being from about 0.5:1.5 to 1.5:0.5.

Particularly preferred is a weight ratio of (a) to (b) from 0.8:1.2 to 1.2:0.8, which imparts a particularly beautiful nacreous character.

Especially recommended for both the formation of the nacreous character and for achieving an unusually high care effect after the dye composition has been rinsed out is a weight ratio of fatty alcohol alkoxylate (c) to ammonium quat (d) from 0.5:1.5 to 1.5:0.5 and particularly from 0.8:1.2 to 1.2:0.8.

Long-chain fatty alcohols suitable according to the invention are, for example, straight-chain or branched fatty alcohols with 8 to 30 carbon atoms and particularly C₁₄-C₂₂ fatty alcohols such as, for example, isooctyl alcohol, tetradecanol, cetyl alcohol, stearyl alcohol, isotridecyl alcohol and behenyl alcohol, as well as mixtures of these fatty alcohols. Suitable fatty alcohols are, among others, also those marketed by Cognis, Germany, under the tradename Lanette®.

Alkanolamides suitable according to the invention are, for example, monoalkanolamides, dialkanolamides or ester amides, preferably the N-acyl derivatives of monoethanolamine or diethanolamine, for example lauric acid monoethanolamide, coco fatty acid monoethanolamide, coco fatty acid diethanolamide, stearic acid monoethanolamide, palmitic acid monoethanolamide or oleic acid monoethanolamide as well as mixture thereof.

Preferably used as fatty alcohol ethoxylates are the ethoxylated fatty alcohols or fatty alcohol polyglycol ethers of the following formula (I)
R—(OCH₂CH₂)ₓ—OH (I)
(wherein R=a straight-chain or branched C₈-C₂₄ alkyl group or a straight-chain or branched C₈-C₂₄ alkenyl group and x=2-300).

Particularly preferred are fatty alcohol alkoxylates (fatty alcohol polyethylene glycol ethers) of formula (I) wherein R denotes a straight-chain or branched C₁₄-C₂₂-alkyl group and x is equal to 2-200, for example the polyethylene glycol ether of stearyl alcohol with 10 to 50 ethylene oxide units in the molecule (for example Steareth-10 or Steareth-20).

Suitable ammonium quats are, in particular, the compounds of formula (II) or (III) 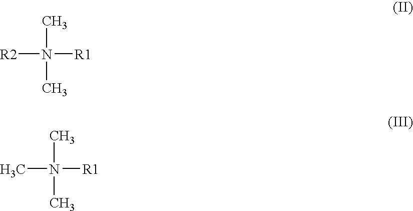
(with R1 and R2 independently of each other denoting a straight-chain or branched C₈-C₂₄-alkyl group).

Preferably used are ammonium quats of formula (II) or (III) wherein R1 and R2 independently of each other denote a straight-chain or branched C₁₄-C₂₂-alkyl group or a mixture thereof, among which distearyldimethylammonium chloride, 2-hexyldecyltrimethylammonium chloride, cetyltrimethylammonium chloride, behenyltrimethylammonium chloride, lauryltrimethylammonium chloride, tetradecyltrimethylammonium chloride, lauryloxy-2-hydroxypropyltrimethylammonium chloride, cocoyltrimethylammonium chloride, soyatrimethylammonium chloride and stearyltrimethylammonium chloride as well as mixtures of these compounds are especially preferred.

Particularly preferred from a standpoint of high storage stability are agents wherein the alkyl chains of the fatty alcohol (a), fatty alcohol alkoxylate (c) and quaternary ammonium compound (d) components are of the same length (for example, the fatty alcohol as well as the fatty alcohol alkoxylate and the ammonium quat contain as the alkyl group a cetyl or stearyl group, the ammonium quat possibly having two cetyl or stearyl groups).

The colorant of the invention is preferably free of common nacreous luster-imparting agents.

The colorant of the invention preferably contains oxidation dye precursors which produce the color by reaction with oxidants, for example hydrogen peroxide, or in the presence of atmospheric oxygen.

Suitable oxidation dye precursors are, for example, the following developers and couplers and self-coupling compounds.

(i) Developers-1,4-diaminobenzene (p-phenylenediamine), 1,4-diamino-2-methylbenzene (p-toluylenediamine), 1,4-diamino-2,6-dimethylbenzene, 1,4-diamino-3,5-diethylbenzene, 1,4-diamino-2,5-dimethylbenzene, 1,4-diamino-2,3-dimethylbenzene, 2-chloro-1,4-diaminobenzene, 1,4-diamino-2-(thiophen-2-yl)benzene, 1,4-diamino-2-(thiophen-3-yl)benzene, 1,4-diamino-2-(pyridin-3-yl)benzene, 2,5-diaminobiphenyl, 1,4-diamino-2-methoxymethylbenzene, 1,4-diamino-2-aminomethylbenzene, 1,4-diamino-2-hydroxymethylbenzene, 1,4-diamino-2-(2-hydroxyethoxy)benzene, 2-[2-(acetylamino)ethoxy]-1,4-diaminobenzene, 4-phenylaminoaniline, 4-dimethylaminoaniline, 4-diethylaminoaniline, 4-dipropylaminoaniline, 4-[ethyl-(2-hydroxyethyl)amino]aniline, 4-[di(2-hydroxyethyl)amino]aniline, 4-[di(2-hydroxyethyl)amino]-2-methylaniline, 4-[(2-methoxyethyl)amino]aniline, 4-[(3-hydroxypropyl)amino]aniline, 4-[(2,3-dihydroxypropyl)amino]aniline, 1,4-diamino-2-(2-hydroxyethyl)benzene, 1,4-diamino-2-(1-methylethyl)benzene, 1,3-bis-[(4-aminophenyl)(2-hydroxyethyl)amino]-2-propanol, 1,4-bis-[(4-aminophenyl)amino]butane, 1,8-bis-(2,5-diaminophenoxy)-3,6-dioxaoctane, 4-aminophenol, 4-amino-3-methylphenol, 4-amino-3-(hydroxymethyl)phenol, 4-amino-3-fluorophenol, 4-methylaminophenol, 4-amino-2-(aminomethyl)phenol, 4-amino-2-(hydroxymethyl)phenol, 4-amino-2-fluorophenol, 4-amino-2-[(2-hydroxyethyl)amino]methylphenol, 4-amino-2-methylphenol, 4-amino-2-(methoxymethyl)phenol, 4-amino-2-(2-hydroxyethyl)phenol, 5-aminosalicylic acid, 2,5-diaminopyridine, 2,4,5,6-tetraminopyrimidine, 2,5,6-triamino-4-(1H)-pyrimidone, 4,5-diamino-1-(2-hydroxyethyl)-1H-pyrazole, 4,5-diamino-1-(1-methylethyl)-1H-pyrazole, 4,5-diamino-1-[(4-methylphenyl)methyl]-1H-pyrazole, 1-[(4-chlorophenyl)methyl]-4,5-diamino-1H-pyrazole, 4,5-diamino-1-methyl-1H-pyrazole, 4,5-diamino-1-pentyl-1H-pyrazole, 4,5-diamino-1-(phenylmethyl)-1H-pyrazole, 1,2-bis-(4,5-diamino-1H-pyrazol-1-yl)ethane, 4,5-diamino-1-[(4-methoxyphenyl)methyl]-1H-pyrazole, 2-aminophenol, 2-amino-6-methyl-phenol, 2-amino-5-methyl-phenol and the diaminopyrazole derivatives mentioned in WO 02/46165 A1, DE 101 09 807 A1 and EP 0 740 931 A1 used alone or in admixture with one another.

(ii) Couplers N-(3-dimethylaminophenyl)urea, 2,6-diaminopyridine, 2-amino-4-[(2-hydroxyethyl)amino]anisole, 2,4-diamino-1-fluoro-5-methylbenzene, 2,4-diamino-1-methoxy-5-methylbenzene, 2,4-diamino-1-ethoxy-5-methylbenzene, 2,4-diamino-1-(2-hydroxyethoxy)-5-methylbenzene, 2,4-di[(2-hydroxyethyl)amino]-1,5-dimethoxybenzene, 2,3-diamino-6-methoxypyridine, 3-amino-6-methoxy-2-(methylamino)pyridine, 2,6-diamino-3,5-dimethoxypyridine, 3,5-diamino-2,6-dimethoxypyridine, 1,3-diaminobenzene, 2,4-diamino-1-(2-hydroxyethoxy)benzene, 1,3-diamino-4-(2,3-dihydroxypropoxy)benzene, 2,4-diamino-1,5-di(2-hydroxyethoxy)benzene, 1-(2-aminoethoxy-2,4-diaminobenzene, 2-amino-1-(2-hydroxyethoxy)-4-methylaminobenzene, 2,4-diaminophenoxyacetic acid, 3-[di(2-hydroxyethyl)amino]aniline, 4-amino-2-di[(2-hydroxyethyl)amino]-1-ethoxybenzene, 5-methyl-2-(1-methylethyl)phenol, 3-[(2-hydroxyethyl)amino]aniline, 3-[(2-aminoethyl)amino]aniline, 1,3-di(2,4-diaminophenoxy)propane, di(2,4-diaminophenoxy)methane, 1,3-diamino-2,4-dimethoxybenzene, 2,6-bis-(2-hydroxyethyl)aminotoluene, 4-hydroxyindole, 3-dimethylaminophenol, 3-diethylaminophenol, 5-amino-2-methylphenol, 5-amino-4-fluoro-2-methylphenol, 5-amino-4-methoxy-2-methylphenol, 5-amino-4-ethoxy-2-methylphenol, 3-amino-2,4-dichlorophenol, 5-amino-2,4-dichlorophenol, 3-amino-2-methylphenol, 3-amino-2-chloro-6-methylphenol, 3-aminophenol, 2-[(3-hydroxyphenyl)amino]acetamide, 5-[(2-hydroxyethyl)amino]-4-methoxy-2-methylphenol, 5-[(2-hydroxyethyl)amino]-2-methylphenol, 3-[(2-hydroxyethyl)amino]phenol, 3-[(2-methoxyethyl)amino]phenol, 5-amino-2-ethylphenol, 5-amino-2-methoxyphenol, 2-(4-amino-2-hydroxyphenoxy)ethanol, 5-[(3-hydroxypropyl)amino]-2-methylphenol, 3-[(2,3-dihydroxypropyl)amino]-2-methylphenol, 3-[(2-hydroxyethyl)amino]-2-methylphenol, 2-amino-3-hydroxypyridine, 5-amino-4-chloro-2-methylphenol, 1-naphthol, 2-methyl-1-naphthol, 1,5-dihydroxynaphthalene, 1,7-dihydroxynaphthalene, 2,3-dihydroxynaphthalene, 2,7-dihydroxynaphthalene, 2-methyl-1-naphthol acetate, 1,3-dihydroxybenzene, 1-chloro-2,4-dihydroxybenzene, 2-chloro-1,3-dihydroxybenzene, 1,2-dichloro-3,5-dihydroxy-4-methylbenzene, 1,5-dichloro-2,4-dihydroxybenzene, 1,3-dihydroxy-2-methylbenzene, 3,4-methylenedioxyphenol, 3,4-methylenedioxyaniline, 5-[(2-hydroxyethyl)amino]-1,3-benzodioxole, 6-bromo-1-hydroxy-3,4-methylenedioxybenzene, 3,4-diaminobenzoic acid, 3,4-dihydro-6-hydroxy-1,4(2H)benzoxazine, 6-amino-3,4-dihydro-1,4(2H)-benzoxazine, 3-methyl-1-phenyl-5-pyrazolone, 5,6-dihydroxyindole, 5,6-dihydroxyindoline, 5-hydroxyindole, 6-hydroxyindole, 7-hydroxyindole and 2,3-indolinedione alone or in admixture with one another.

(iii) Self-coupling compounds: 2-amino-5-methylphenol, 2-amino-6-methylphenol, 2-amino-5-ethoxyphenol or 2-propylamino-5-aminopyridine.

Naturally, the oxidation dye precursors, provided they are bases, can also be used in the form of their physiologically compatible salts of organic or inorganic acids, for example hydrochloric acid or sulfuric acid, or—if they contain aromatic OH— groups—in the form of salts of bases, for example as alkali metal phenoxides.

The total amount of the oxidation dye precursors contained in the colorant of the invention is about 0.01 to 12 weight percent and particularly about 0.2 to 6 weight percent.

For the purpose of achieving certain color shades, the aforesaid oxidation dyes can contain besides the oxidation dye precursors also naturally occurring and/or synthetic direct dyes.

Suitable natural or synthetic direct dyes are, for example, the vegetable dyes such as henna or indigo, furthermore triphenylmethane dyes, aromatic nitro dyes, azo dyes, quinone dyes, disperse dyes and cationic or anionic dyes.

The total amount of direct dyes in the colorant of the invention is about 0.01 to 7 weight percent and preferably about 0.2 to 4 weight percent.

Other common dyes known to be used for hair dyeing and which can be contained in the colorant of the invention are described in, among others, E. Sagarin, “Cosmetics, Science and Technology”, Interscience Publishers, Inc., New York (1957), pages 503 ff, in H. Janistyn, “Handbuch der Kosmetika und Riechstoffe” [Handbook of Cosmetics and Fragrance Materials], vol. 3 (1973), pages 388 ff, and in K. Schrader “Grundlagen und Rezepturen der Kosmetika” [Fundamentals and Formulations of Cosmetics], 2nd edition (1989), pages 782-815.

Although oxidation dyes are preferred, it is, of course, also possible for the colorant of the invention to be in the form of a nonoxidative colorant based on the aforesaid direct dyes.

The total amount of direct dyes in these nonoxidative colorants is about 0.01 to 10 weight percent and preferably about 0.2 to 5 weight percent.

Moreover, if the colorants are used for dyeing hair, they can also contain other common cosmetic additives for example antioxidants such as ascorbic acid, thioglycolic acid or sodium sulfite, as well as perfume oils, complexing agents, for example an ethylenediaminetetraacetate or nitriloacetic acid, furthermore wetting agents, emulsifiers, thickeners and hair-care agents, the perfume oils being used in an amount from about 0.001 to 1 weight percent, and the antioxidants and complexing agents each being used in an amount from about 0.001 to 0.5 weight percent.

Naturally, the afore-described colorant can optionally contain other additives commonly used in colorants, for example solvents such as water, lower aliphatic alcohols, for example ethanol, n-propanol and isopropanol, or glycols such as glycerol and 1,2-propanediol, furthermore (in addition to the aforesaid fatty alcohol alkoxylates and ammonium quats) wetting agents or emulsifiers from the classes of anionic, cationic, amphoteric or nonionic surfactants, such as the fatty alcohol sulfates, ethoxylated fatty alcohol sulfates, alkylsulfonates, alkylbenzenesulfonates, alkyltrimethylammonium salts, alkylbetaines, ethoxylated fatty alcohols, ethoxylated nonylphenols, ethoxylated fatty alcohols [sic], ethoxylated nonylphenols [sic], ethoxylated fatty esters, furthermore thickeners such as starch, cellulose derivatives, perfumes, hair pretreatment agents, conditioners, hair-swelling agents, preservatives, moreover vaselines, paraffin oil and fatty acids as well as hair-care agents such as cationic resins, lanolin derivatives, cholesterol, pantothenic acid and betaine. The said constituents are used in amounts commonly employed for such purposes, for example the wetting agents and emulsifiers at a concentration from about 0.1 to 30 weight percent, and the hair-care agents at a concentration from about 0.1 to 5.0 weight percent.

The pH of the colorants of the invention in the case of oxidative colorants based on oxidation dye precursors is in the range from about 6 to 12 and preferably from 9 to 11, whereas the pH of the ready-to-use oxidation colorant (namely the mixture of the colorant of the invention and the oxidant) is about 5.5 to 10 and preferably 6 to 9. For nonoxidative colorants based on direct dyes, the pH is in the range from about 5 to 10 and preferably 6 to 9.

Depending on the composition and the desired pH of the colorant, the pH is preferably adjusted with ammonia or an organic amine, for example a glucamine, aminomethylpropanol, monoethanolamine or triethanolamine, an inorganic base, for example sodium hydroxide, potassium hydroxide, sodium carbonate or calcium hydroxide, or with an organic or inorganic acid such as, for example, lactic acid, citric acid, acetic acid or phosphoric acid.

The colorant of the invention is preferably formulated as an aqueous or aqueous-alcoholic preparation, for example as a thickened solution or as an emulsion, cream or gel.

For use in oxidative dyeing, the afore-described colorant is mixed with an oxidant just before use, and the mixture is applied to the fibers in an amount sufficient for dyeing, as a rule about 60 to 200 grams of the ready-to-use preparation.

Suitable oxidants for developing the color are mainly hydrogen peroxide or the compounds of addition thereof to urea, melamine or sodium borate in the form of a 1 to 12% and preferably 1.5 to 6% aqueous solution. The mixing ratio of colorant to oxidant is as a rule from about 5:1 to 1:5, preferably from 2:1 to 1:3 and particularly 1:1, the amount of oxidant in the ready-to-use preparation preferably being from about 0.5 to 8 weight percent and particularly from 1 to 4 weight percent.

If the colorant of the invention contains no oxidation dye precursors (nonoxidative colorant) or contains oxidation dye precursors that are readily oxidized by atmospheric oxygen, the colorant can be applied directly to the keratin fibers without previous mixing with an oxidant. It is also possible, however, in order to achieve simultaneous brightening of the fibers or faster oxidation of the dyes, to mix these agents with an oxidant before use.

The ready-to-use colorant is allowed to act on the fibers (for example human hair) at about 15 to 50° C. for about 10 to 45 minutes, preferably for about 15 to 30 minutes, after which the fibers are rinsed with water and dried. Following this rinsing, the fibers can be washed with a shampoo and optionally post-rinsed with a weak organic acid, for example tartaric acid. The fibers are then dried.

A colorant prepared so as to have the composition of the invention meets requirements in terms of adhesion properties, application properties and viscosity adjustment in outstanding manner. In addition, the hair effect achieved with the colorant of the invention after rinsing is appreciably better than with the prior-art colorants. Moreover, the colorants of the invention have a uniform consistency and a nacreous luster (“nacreous effect”) that is cosmetically very attractive.

The following examples are intended to illustrate the subject matter of the invention in greater detail without limiting its scope.

### EXAMPLES

EXAMPLE 1
Oxidative Hair Colorant, Creamy

| | | |
|---|---|---|
| 6.0000 | g | of stearyl alcohol |
| 5.0000 | g | of cetyl alcohol |
| 8.0000 | g | of coco fatty acid monoethanolamide |
| | | (Oramide ® ML 115, |
| | | supplied by Seppic/FR) |
| 4.0000 | g | of Steareth-20 (Volpo ® S20, |
| | | supplied by Croda/GB) |
| 5.0000 | g | of stearyltrimethylammonium chloride |
| | | (Arquad ® 18-50, |
| | | supplied by Akzo Nobel/FR) |
| 1.3620 | g | of 4-aminophenol |
| 0.5000 | g | of 1-naphthol |
| 0.0136 | g | of resorcinol |
| 0.0034 | g | of 2-amino-6-chloro-4-nitrophenol |
| 12.0000 | g | of ammonia, 25% aqueous solution |
| 1.0000 | g | of disodium ethylenediaminetetraacetate |
| 1.0000 | g | of ascorbic acid |
| to 100.0000 | g | water |

Just before use, 50 g of the above hair colorant with a nacreous luster was mixed with 50 g of a 6% aqueous hydrogen peroxide solution. This gave a uniform, cosmetically attractive colorant preparation with a nacreous luster. The mixture thus obtained was then applied to naturally blond hair. After an exposure time of 30 min at 40° C., the hair was rinsed with water and dried. The hair had a lustrous copper-red color.

EXAMPLE 2
Creamy, Brightening Oxidation Hair Colorant

| Component (A): | | Creamy Dye Carrier Composition |
|---|---|---|
| 6.00 | g | of behenyl alcohol |
| 8.00 | g | of stearyl alcohol |
| 8.00 | g | of coco fatty acid monoethanolamide |
| | | (Comperlan ® 100, supplied |
| | | by Cognis/DE) |
| 6.00 | g | of Steareth-10 (Brij ® 76, supplied |
| | | by ICI Surfactants) |
| 1.00 | g | of oleic acid |
| 4.00 | g | of behenyltrimethylammonium chloride |
| | | (Genamin ® KDMP, |
| | | supplied by Clariant/CH) |
| 0.50 | g | of p-phenylenediamine |
| 0.07 | g | of resorcinol |
| 1.00 | g | of disodium ethylenediaminetetraacetate |
| 8.00 | g | of ammonia, 25% aqueous solution |
| 8.00 | g | of ethanol |
| to 100.00 | g | water |
| Component (B): | | Hydrogen Peroxide Emulsion |
| 10.0 | g | of cetylstearyl alcohol |
| 1.5 | g | of cholesterol |
| 4.0 | g | of sodium lauryl alcohol diethylene glycol ether |
| | | sulfate, 25% aqueous solution |
| 35.0 | g | of hydrogen peroxide, 35% aqueous solution |
| 0.3 | g | of perfume |
| to 100.0 | g | water |

Before use, 40 g of the liquid nacreous dye carrier composition (A) was mixed with 80 g of the hydrogen peroxide emulsion (B) in an (A): (B) mixing ratio of 1:2, and 120 g of the resulting nacreous mixture was applied to gray human hair. After an exposure time of 20 min at room temperature, the hair was rinsed with water and dried. The hair thus treated had a uniform light-brown color from the hair roots to the hair tips. The colorant of the invention was easy to apply and did not run off the hair.

EXAMPLE 3
Oxidative Hair Colorant, Creamy

| | | |
|---|---|---|
| 4.00 | g | of cetylstearyl alcohol |
| 5.00 | g | of behenyl alcohol |
| 12.00 | g | of coco fatty acid monoethanolamide |
| | | (Rewomid ® C 212, |
| | | supplied by Goldschmidt/DE) |
| 2.00 | g | of Steareth-20 (Alkanol S20P, supplied |
| | | by Goldschmidt) |
| 5.00 | g | of distearyldimethylammonium chloride |
| | | (Arquad ® 2HAT-75, |
| | | supplied by Akzo Nobel) |
| 8.00 | g | of monoethanolamine |
| 1.30 | g | of 1-methyl-2,5-diaminobenzene |
| 1.00 | g | of beeswax |
| 0.65 | g | of resorcinol |
| 0.50 | g | of keratin hydrolyzate |
| 0.50 | g | of silk protein hydrolyzate |
| 0.52 | g | of 2-amino-6-chloro-4-nitrophenol |
| 1.00 | g | of disodium ethylenediaminetetraacetate |
| 0.30 | g | of ascorbic acid |
| to 100.00 | g | water |

Just before use, 50 g of the above colorant with a nacreous luster was mixed with 50 g of a 12% aqueous hydrogen peroxide solution. The nacreous luster was retained even after the mixing. The mixture was then applied to naturally blond hair and, after an exposure time of 30 min at 40° C., the mixture was rinsed out of the hair. After drying, the hair had a uniform, deep brown shade.

EXAMPLE 4
Nonoxidative Hair Colorant

| | | |
|---|---|---|
| 6.000 | g | of stearyl alcohol |
| 5.000 | g | of behenyl alcohol |
| 8.000 | g | of coco fatty acid monoethanolamide |
| | | (Rewomid ® C 212, |
| | | supplied by Goldschmidt/DE) |
| 2.000 | g | of Steareth-20 (Volpo ® S20, supplied |
| | | by Croda/GB) |
| 2.000 | g | of distearyldimethylammonium chloride |
| 2.000 | g | of isopropyl alcohol |
| 0.160 | g | of 4-[ethyl(2-hydroxyethyl)amino]-1-[(2- |
| | | hydroxyethyl)amino]-2-nitro- |
| | | benzene hydrochloride (HC Blue No. 12) |
| 0.170 | g | of 4-[(2,3-dihydroxypropyl)amino]-3-nitro- |
| | | 1-trifluoromethylbenzene |
| | | (HC Yellow No. 6) |
| 0.012 | g | of 1-N-hydroxyethylamino-4-methyl-2-nitrobenzene |
| 0.025 | g | of 1-amino-5-chloro-4-[(2,3-dihydroxypropyl)amino]- |
| | | 2-nitrobenzene |
| | | (HC Red No. 10) |
| 0.010 | g | of 5-chloro-1,4-[di(2,3-dihydroxypropyl)amino]- |
| | | 2-nitrobenzene |
| | | (HC Red No. 11) |
| to 100.00 | g | water |

The nacreous, creamy dye composition was applied to washed, towel-dried, natural blond hair and allowed to act for about 20 to 25 minutes. Excess dye was then washed out with water and a shampoo, and the hair was dried and optionally styled. A beautiful, lustrous, medium-blond shade was obtained.

EXAMPLE 5
Nonoxidative Hair Colorant

| | | |
|---|---|---|
| 3.00 | g | of cetylstearyl alcohol |
| 3.00 | g | of stearyl alcohol |
| 5.00 | g | of coco fatty acid diethanolamide |
| | | (Rewomid ® C 212 S, |
| | | supplied by Goldschmidt/DE) |
| 2.00 | g | of Oleth-30 |
| 2.50 | g | of behenyltrimethylammonium chloride |
| 7.00 | g | of ethanol |
| 0.10 | g | of 1-N-hydroxyethylamino-4-methyl-2-nitrobenzene |
| 0.35 | g | of 1-amino-5-chloro-4-[(2,3-dihydroxypropyl)amino]- |
| | | 2-nitrobenzene |
| | | (HC Red No. 10) |
| 0.15 | g | of 5-chloro-1,4-[di(2,3-dihydroxypropyl)amino]- |
| | | 2-nitrobenzene |
| | | (HC Red No. 11) |
| 0.20 | g | of 2-amino-6-chloro-4-nitrophenol |
| to 100.00 | g | water |

The nacreous, creamy dye composition was applied to washed, towel-dried, natural blond hair and allowed to act for about 20 to 25 minutes. Excess dye was then washed out with water and a shampoo, and the hair was dried and optionally styled. A beautiful, lustrous, fashionable red shade was obtained.

EXAMPLE 6
Hair Colorant

| | | |
|---|---|---|
| 0.33 | g | of 2,5-diaminotoluene sulfate |
| 0.33 | g | of 2,5-diaminophenylethanol sulfate |
| 0.22 | g | of N,N-bis-(β-hydroxyethyl)-p-phenylenediamine sulfate |
| 0.33 | g | of 4-amino-3-methylphenol |
| 0.22 | g | of 2-aminomethyl-p-aminophenol.HCl |
| 0.05 | g | of 4,5-diamino-1-hydroxyethylpyrazole sulfate |
| 0.05 | g | of 4,5-diamino-1-(p-methylbenzyl)pyrazole sulfate |
| 0.05 | g | of 4,5-diamino-1-(1-methylethyl)pyrazole sulfate |
| 0.05 | g | of 4,5-diamino-1-methylpyrazole sulfate |
| 0.05 | g | of 4,5-diamino-1-(4-methoxybenzyl)pyrazole sulfate |
| 0.05 | g | of 4,5-diamino-1-benzylpyrazole sulfate |
| 0.05 | g | of 1,2-bis(4,5-diamino-1H-pyrazol-1-yl)ethane sulfate |
| 0.05 | g | of 4,5-diamino-1-(4-chlorobenzyl)pyrazole sulfate |
| 0.05 | g | of 4,5-diamino-1-pentylpyrazole sulfate |
| 0.11 | g | of 1-naphthol |
| 0.11 | g | of 3,4-diaminobenzoic acid |
| 0.11 | g | of 1-(β-hydroxyethylamino)-3,4- |
| | | methylenedioxybenzene.HCl |
| 0.11 | g | of 2,4-diamino-1-(β-hydroxyethoxy)benzene sulfate |
| 0.11 | g | of 5-amino-6-chloro-2-methylphenol |
| 0.11 | g | of 1,3-bis-2,4-(diaminophenoxy)propane.2 HCl |
| 0.11 | g | of 3-aminophenol |
| 0.11 | g | of 4-chlororesorcinol |
| 0.11 | g | of 5-amino-2-methylphenol |
| 0.11 | g | of 2-amino-4-(β-hydroxyethylamino)anisole sulfate |
| 0.11 | g | of 2,4-diamino-1-fluoro-5--methylbenzene sulfate (1:1) |
| 0.11 | g | of 3,5-diamino-2,6-dimethoxypyridine.2 HCl |
| 0.11 | g | of resorcinol |
| 0.11 | g | of 2-methylresorcinol |
| 0.11 | g | of m-dimethylaminophenylurea |
| 0.02 | g | of 2-amino-5-methylphenol |
| 0.02 | g | of 2-amino-6-chloro-4-nitrophenol |
| 0.02 | g | of 4-(β-hydroxyethylamino)-3-nitrophenol |
| 0.02 | g | of 4-(3-hydroxypropylamino)-3-nitrophenol |
| 0.02 | g | of N-1-(2-hydroxyethyl)-2-nitro-p-phenylenediamine |
| | | (HC Red No. 3) |
| 0.02 | g | of 4-amino-3-nitrophenol |
| 0.02 | g | of 2-amino-4,6-dinitrophenol, 65% aqueous solution |
| 0.02 | g | of 2-hydroxyethylpicramic acid |
| 0.02 | g | of 1-N-hydroxyethylamino-4-methyl-2-nitrobenzene |
| 0.02 | g | of 6-ethylamino-2-chloro-4-nitrophenol |
| 0.02 | g | of 2,6-diamino-3-[(pyridin-3-yl)azo]pyridine |
| 0.02 | g | of 4-nitrophenylaminoethylurea |
| 0.01 | g | of 1-amino-5-chloro-[(2,3-dihydroxypropyl)amino]- |
| | | 2-nitrobenzene |
| 4.00 | g | of cetylstearyl alcohol |
| 5.00 | g | of behenyl alcohol |
| 12.00 | g | of coco fatty acid monoethanolamide |
| 2.00 | g | of Steareth-20 |
| 5.00 | g | of distearyldimethylammonium chloride |
| 8.00 | g | of monoethanolamine |
| 3.50 | g | of ammonia, 25% aqueous solution |
| 1.00 | g | of beeswax |
| 0.50 | g | of keratin hydrolyzate |
| 0.50 | g | of silk protein hydrolyzate |
| 1.00 | g | of disodium ethylenediaminetetraacetate |
| 0.30 | g | of ascorbic acid |
| to 100.00 | g | water |

Just before use, 50 g of the above colorant with a nacreous luster was mixed with 50 g of a 12% aqueous hydrogen peroxide solution. The nacreous luster was retained even after the mixing. The mixture was then applied to naturally blond hair and, after an exposure time of 30 min at 40° C., the mixture was rinsed out of the hair. After drying, the hair had a uniform, deep black-brown shade.

Unless otherwise indicated, all percentages given in the present patent application are by weight.

1-20. (canceled)

21. Colorant for dyeing keratin fibers containing oxidation dye precursors and/or direct dyes, said colorant comprising a combination of at least one long-chain fatty alcohol, at least one alkanolamide, at least one fatty alcohol alkoxylate and at least one quaternary ammonium compound;
wherein a weight ratio of said at least one long-chain fatty alcohol to said at least one alkanolamide is from 0.5:1.5 to 1.5:0.5.

22. Colorant for dyeing keratin fibers containing oxidation dye precursors and/or direct dyes, said colorant comprising a combination of from 2 to 12 percent by weight of at least one long-chain fatty alcohol, from 2 to 12 percent by weight of at least one alkanolamide, from 0.5 to 10 percent by weight of at least one fatty alcohol alkoxylate and from 0.5 to 10 percent by weight of at least one quaternary ammonium compound;
wherein a weight ratio of said at least one long-chain fatty alcohol to said at least one alkanolamide is from 0.5:1.5 to 1.5:0.5.

23. Colorant as defined in claim 21 or 22, wherein said weight ratio is from 0.8:1.2 to 1.2:0.8.

24. Colorant as defined in claim 21 or 22, wherein a weight ratio of said at least one fatty alcohol alkoxylate to said at least one quaternary ammonium compound is from 0.5:1.5 to 1.5:0.5.

25. Colorant as defined in claim 21 or 22, wherein a weight ratio of said at least one fatty alcohol alkoxylate to said at least one quaternary ammonium compound is from 0.8:1.2 to 1.2:0.8.

26. Colorant as defined in claim 21 or 22, wherein said at least one long-chain fatty alcohol is a straight-chain or branched fatty alcohol with 8 to 30 carbon atoms.

27. Colorant as defined in claim 21 or 22, wherein said at least one long-chain fatty alcohol is selected from the group consisting of cetyl alcohol, stearyl alcohol and behenyl alcohol, or a mixture thereof.

28. Colorant as defined in claim 21 or 22, wherein said at least one alkanolamide is a monoalkanolamide, a dialkanolamide or an ester amide.

29. Colorant as defined in claim 21 or 22, wherein said at least one alkanolamide is an N-acyl derivative of monoethanolamine or diethanolamine.

30. Colorant as defined in claim 21 or 22, wherein said at least one alkanolamide is selected from the group consisting of lauric acid monoethanolamide, coco fatty acid monoethanolamide, coco fatty acid diethanolamide, stearic acid monoethanolamide, palmitic acid monoethanolamide and oleic acid monoethanolamide, or a mixture of said monoethanolamides.

31. Colorant as defined in claim 21 or 22, wherein said at least one fatty alcohol alkoxylate is a fatty alcohol polyglycol ether of formula (I)
R—(OCH₂CH₂)ₓ—OH (I),
wherein R is a straight-chain or branched C₈-C₂₄-alkyl group or a straight-chain or branched C₈-C₂₄-alkenyl group and x equals 2 to 300.

32. Colorant as defined in claim 21 or 22, wherein said at least one fatty alcohol alkoxylate is a polyethylene glycol ether of stearyl alcohol with 10 to 50 ethylene oxide units.

33. Colorant as defined in claim 21 or 22, wherein said at least one quaternary ammonium compound is a compound of formula (II) or (III): 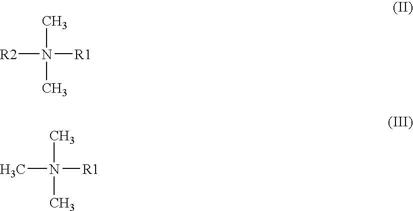
wherein R1 and R2, independently of each other, denote a straight-chain or branched C₈-C₂₄-alkyl group.

34. Colorant as defined in claim 21 or 22, wherein said at least one quaternary ammonium compound is selected from the group consisting of distearyldimethyl ammonium chloride, 2-hexyldecyltrimethyl ammonium chloride, cetyl trimethyl ammonium chloride, behenyl-trimethyl ammonium chloride, lauryltrimethyl ammonium chloride, lauryloxy-2-hydroxy-propyltrimethyl ammonium chloride, cocoyltrimethyl ammonium chloride, soyatrimethyl ammonium chloride and stearyltrimethyl ammonium chloride, or a mixture of said ammonium chlorides.

35. Colorant as defined in claim 21 or 22, containing an oxidation dye and from 0.01 to 12 weight percent of at least one of said oxidation dye precursors.

36. Colorant as defined in claim 21 or 22, containing from 0.01 to 7 weight percent of at least one of said direct dyes.

37. Colorant as defined in claim 21 or 22, consisting of a non-oxidative colorant and containing from 0.01 to 10 weight percent of at least one of said direct dyes.

38. Colorant as defined in claim 21 or 22, consisting of a hair colorant.

39. A method of making a colorant with a nacreous luster for keratin fibers, said method comprising combining oxidation dye precursors and/or direct dyes with a combination of at least one long-chain fatty alcohol, at least one alkanolamide, at least one fatty alcohol alkoxylate and at least one quaternary ammonium compound, so that a weight ratio of said at least one long-chain fatty alcohol to said at least one alkanolamide is from 0.5:1.5 to 1.5:0.5.

40. A method of making a colorant with a nacreous luster for keratin fibers, said method comprising combining oxidation dye precursors and/or direct dyes with a combination of from 2 to 20 percent by weight of at least one long-chain fatty alcohol, from 2 to 12 percent by weight of at least one alkanolamide, from 0.5 to 10 percent by weight of at least one fatty alcohol alkoxylate and from 0.5 to 10 percent by weight of at least one quaternary ammonium compound, so that a weight ratio of said at least one long-chain fatty alcohol to said at least one alkanolamide is from 0.5:1.5 to 1.5:0.5.

41. The method as defined in claim 39 or 40, wherein said weight ratio is from 0.8:1.2 to 1.2:0.8.

42. The method as defined in claim 39 or 40, wherein a weight ratio of said at least one fatty alcohol alkoxylate to said at least one quaternary ammonium compound is from 0.5:1.5 to 1.5:0.5.

43. The method as defined in claim 39 or 40, wherein a weight ratio of said at least one fatty alcohol alkoxylate to said at least one quaternary ammonium compound is from 0.8:1.2 to 1.2:0.8.
